(19) United States
(12) Patent Application Publication
(10) Pub. No.: US 2011/0070440 A1
(43) Pub. Date: Mar. 24, 2011

(54) Artificial Organelle On A Digital Microfluidic Chip Used To Redesign The Biological Activities of Heparan Sulfate

(76) Inventors: Robert J. Linhardt, Albany, NY (US); Jonathan S. Dordick, Schenectady, NY (US)

(21) Appl. No.: 12/831,886

(22) Filed: Jul. 7, 2010

Related U.S. Application Data
(60) Provisional application No. 61/223,575, filed on Jul. 7, 2009.

(51) Int. Cl. C25B 7/00; C08B 37/10; C07K 14/00; B32B 5/16; B82Y 5/00

(52) U.S. Cl. 428/402; 204/451; 536/21; 530/395; 977/773

(57) ABSTRACT

Using digital microfluidics, recombinant enzyme technology, and magnetic nanoparticles, a functional prototype of an artificial Golgi organelle is described. Analogous to the natural Golgi, which is responsible for the enzymatic modification of glycosaminoglycans immobilized on proteins, this artificial Golgi enzymatically modifies glycosaminoglycans, such as heparan sulfate (HS) chains, immobilized onto magnetic nanoparticles. Sulfo groups were transferred from adenosine 3′-phosphate 5′-phosphosulfate to the 3-hydroxyl group of the D-glucosamine residue in an immobilized HS chain using D-glucosaminyl 3-O-sulfotransferase. After modification, the nanoparticles with immobilized HS exhibited increased affinity for fluorescently labeled antithrombin III as detected by confocal microscopy. Since the biosynthesis of HS involves an array of specialized glycosyl transferases, epimerase, and sulfotransferases, this approach should mimic the synthesis of HS in vivo. Furthermore, our method demonstrates the feasibility of investigating the effects of multi-enzyme systems on the structure of final glycan products for HS-based glycomic studies.

### BACKGROUND OF THE INVENTION

The Golgi organelle, discovered in 1898 by Camillo Golgi, remains one of the most poorly understood organelles in the human cell. This organelle is central to the posttranslational modification of proteins, the new frontier in the field of glycomics. It is estimated that the majority of human proteins are posttranslationally modified within the Golgi,¹ primarily through glycosylation, and that these modifications are critical for protein function and stability.

Heparan sulfate (HS) glycosylation of proteoglycans (PGs) takes place in the Golgi organelle (FIG. 1a). Linear, O-linked glycosaminoglycan (GAG) chains composed of repeating glucuronic acid and N-acetylglucosamine residues are attached to the serine residues of a core protein through a linkage region to form a PG and are sequentially modified by a series of biosynthetic enzymes including an N-deacetylase/N-sulfotransferase (NDST), C₅ epimerase, and 2-, 6-, and 3-O-sulfotransferases (2-OST, 6-OST and 3-OST, respectively). NDST catalyzes the N-deacetylation/N-sulfonation of the glucosamine residues, C₅ epimerase converts glucuronic acid into iduronic acid, and 2-OST, 6-OST, and 3-OST catalyze the O-sulfonation of the 2-O-position of the uronic acid, and the 6-O and 3-O-positions of the glucosamine unit, respectively.^{2,3} There is also an alternative theory of GAG biosynthesis that suggests the concerted action between one or more of the biosynthetic enzymes.⁴

Many of these modifications in the Golgi are only partially complete, resulting in a large number of sequence permutations, dramatically complicating the structure of HS.⁴ The ensemble of the GAG chains afforded contain different and often unique structural features that determine their protein binding specificity and ultimate biological activity. PGs synthesized with specific GAG chains are found in the extracellular matrix, and on the surface of mammalian cells. They are involved in important physiological and pathophysiological processes including blood coagulation, infection, cellular growth and differentiation, tumor metastasis, and angiogenesis.⁵

It is believed that the structure of the Golgi apparatus contributes to the structure of HS. Unfortunately, the complexity of the Golgi apparatus limits our understanding of HS biosynthesis. Because studying the in vitro enzymatic synthesis of HS in the absence of a Golgi structure cannot permit a full understanding of the control and regulation of HS biosynthesis, we undertook the design and fabrication of an artificial Golgi using a digital microfluidic platform (FIG. 1b) that resembles the natural organelle. This work represents the first step toward the creation of such a biomimetic organelle.

Microfluidics and lab-on-a-chip technologies enable reactions on the micro- and nanoscales, reducing reagent consumption and analysis time, increasing reaction control and throughput, and providing opportunities for full automation.⁶ Two types of microfluidic systems have been developed: 1) channel microfluidics, which involves fluid flow in patterned channels,; and 2) digital microfluidics, wherein open droplet movement occurs through the process of electrowetting on a 2D grid-like platform. Digital microfluidics has gained popularity by eliminating many of the constraints associated with fixed channels⁷ and allowing individual droplets in a biochemical array to be addressed. Some previous applications of digital microfluidics include glucose and other enzyme-based assays, preparation of protein samples for matrix-assisted laser desorption/ionization mass spectrometry, polymerase chain reaction, and cell-based assays.⁸

Digital microfluidics chips generally comprise an array of electrodes (FIG. 1c) coated with an insulator followed by a hydrophobic layer (FIG. 1d). Droplet movement in digital microfluidics is driven by electrowetting, the ability of a surface to tune its wettability by the application of electrical pulses. To operate a digital microfluidic device, a droplet of fluid is placed over one electrode and then a voltage is applied to an adjacent electrode, causing the insulator above that electrode to become charged. This makes the destination electrode more hydrophilic causing the droplet to move.⁹ This wettability of the surface is reversible, thus allowing the droplet to be moved to an adjacent electrode of choice. In this manner, sample-containing droplets may be transported, mixed, and separated on the chip. However, the devices known in the art are not well suited for a process as complex as an artificial organelle.

The current study investigates the modification of HS by D-glucosaminyl 3-O-sulfotransferase isoform-1 (3-OST-1) using improved digital microfluidics to afford an HS with an increased affinity for anticoagulant protein antithrombin III (ATIII). This represents a first step towards the construction of an artificial Golgi organelle and controlled biosynthesis of GAGs.

### SUMMARY OF THE INVENTION

The invention relates to improved digital microfluidics devices (also called “microfluidics devices”) characterized by the use of magnetic nanoparticles to transport a substrate from a first reaction site to a second or subsequent reaction site. The substrate is preferably immobilized on the magnetic nanoparticle. At least one, preferably at least two or more, of the reaction sites comprise an enzyme.

The invention includes a method for enzymatically producing a biological compound from a substrate comprising, (1) providing a microfluidics device comprising (a) a support comprising a support surface; (b) an array of drive electrodes disposed on the surface; and (c) an electrode selector for sequentially activating and de-activating one or more selected drive electrodes of the array to sequentially bias the selected drive electrodes to an actuation voltage, whereby a droplet disposed on the substrate surface moves along a desired path defined by the selected drive electrodes;

(2) providing a first droplet comprising a substrate, preferably immobilized on a magnetic nanoparticle;

(3) providing an enzyme composition, preferably in a second droplet;

(4) contacting the first droplet and enzyme composition under conditions suitable for reacting the enzyme composition with the substrate;

(5) optionally separating the magnetic nanoparticles using a magnet; and

(6) activating and/or deactivating said drive electrodes to transport the droplet, or a portion thereof.

The enzyme composition contains an enzyme and any cofactors essential, necessary or desirable to complete the reaction. The reaction time can be controlled manually or automated so that the substrate can be completely or partially reacted, as desired. The method preferably further comprises the steps of providing a second enzyme composition, preferably in a third droplet, and contacting the reaction product of step (4) with the second enzyme composition under conditions suitable for reacting the second enzyme composition with the reaction product.

In one embodiment, the invention relates to an in vitro enzymatic glycosylation reaction in a digital microfluidic device. The glycosylation reaction is preferably used to synthesize proteoglycans, such as glycosaminoglycans, specifically heparan sulfate. The advantages of the invention include:
a. Controlled on-chip glycosylation;
b. Solid phase enzymatic controlled synthesis / modification of glycosaminoglycans;
c. Evaporation issues with other microscale devices such as microarrays have been solved; and
d. Automation capabilities

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1. The Golgi and an artificial Golgi. (a) A cartoon in which the Golgi of a eukaryotic cell is shown. The direction of flow (arrow) in posttranslational modification is from the ER to the cell membrane where proteoglycans and glycoproteins are released into the extracellular environment. (b) The design of an artificial Golgi for the biosynthesis of HS is shown. Heparosan is modified by a series of biosynthetic enzymes including an N-deacetylase/N-sulfotransferase (NDST), C₅ epimerase, and 2-, 6-, and 3-O-sulfotransferases (2-OST, 6-OST and 3-OST, respectively).³ Alternatively heparosan can be modified by using a mixture of enzymes to explore the theory of concerted rather then sequential action of the biosynthetic enzymes. The large boxes are reagents and enzyme reservoir electrodes and the small boxes are electrodes for droplet movement, mixing, and sequestration. (c) A fabricated artificial Golgi based on the design in panel b. The thin lines are gold wires that lead to pads for connection to a power source used to drive the droplets. (d) A diagram showing a cross-section of a droplet sitting over an electrode and overlapping two adjacent electrodes. A glass coverslip on the top of the device contains the ground.

FIG. 2. Chemistry used to immobilize HS substrate onto streptavidin magnetic nanoparticles and subsequent 3-OST-1 modification. Unsubstituted amino groups are first N-acetylated and the resulting HS is then reductively aminated with diaminopyridine to which NHS activated PEG₁₀₄-biotin is attached. The resulting glycoconjungate is then captured by streptavidin magnetic nanoparticles and modified by 3-OST-1, which catalyzes the 3-O-sulfonation of HS using PAPS cofactor. During this reaction, the sulfo group is transferred from the PAPS cofactor to the glucosamine residue on the HS chain. To quantify the level of 3-O-sulfonation, [³⁵S]PAPS was used. Scintillation counting measured the transfer of 1 pmol of 3-O-sulfo groups to every 2 μg of HS when immobilized to nanoparticles.

FIG. 3. Schematic of 3-OST-1 modification of immobilized HS on nanoparticles and subsequent detection with fluorescently labeled ATIII taking place on the device shown in FIG. 1c. A magnet was used to selectively wash the nanoparticles in solution. The contents of the droplets are labeled as followed (a) 3-OST-1 enzyme with PAPS (b) immobilized HS (c) Modification of HS by 3-OST-1 (d) Removal of 3-OST-1, excess PAPS, and PAP (e) Modified HS (f) Fluorescently labeled ATIII (g) Mixing of fluorescent ATIII with modified immobilized HS (h) Excess fluorescently labeled ATIII removed from washing step (i) Fluorescence detection of modified HS on nanoparticles.

FIG. 4. Confocal images and image analysis of HS nanoparticles modified off-chip with 3-OST-1 and probed with fluorescent ATIII and washed. (a) Fluorescence image of ATIII bound to 3-OST-1 modified HS nanoparticles. (b) Transmitted bright field image of 3-OST-1 modified HS nanoparticles (c) Overlay of (a) and (b). (d) Fluorescence area selected by ImageJ from (a) shown as light spots on the dark background. (e) Nanoparticle area selected by ImageJ from (b).

FIG. 5. Confocal images and image analysis of control unmodified HS nanoparticles probed with fluorescent ATIII and washed. (a) Fluorescence image of ATIII bound to 3-OST-1 modified HS nanoparticles. (b) Transmitted bright field image of 3-OST-1 modified HS nanoparticles (c) Overlay of (a) and (b). (d) Fluorescence area selected by ImageJ from (a) shown as light spots on dark background. (e) Nanoparticle area selected by ImageJ from (b).

FIG. 6. Graph comparing the percent coverage of fluorescent ATIII on unmodified HS nanoparticles, 3-OST-1 off-chip modified HS nanoparticles, and 3-OST-1 on-chip modified HS nanoparticles. Both the off-chip and on-chip 3-OST-1 modified HS nanoparticles show a significant increase in ATIII binding affinity when compared to the unmodified control. Error bars are standard deviations based on n=4.

FIG. 7. Video sequence of on-chip enzymatic modification of immobilized HS on nanoparticles by 3-OST-1 enzyme. Time stamps in h:min:sec format. (a-c) Small circular droplet containing 3-OST-1 enzyme and PAPS in PBS buffer (E) is joined with a small circular droplet containing HS substrate immobilized on magnetic nanoparticles (S-B) in 1.13 s. (d-f) Large oval droplet containing reaction components are mixed by stretching. (g-i) Large oval droplet can be split into equal sized smaller circular droplets with one taken off chip for analysis and the other used in secondary reactions.

FIG. 8. Confocal images and image analysis of HS nanoparticles modified on-chip with 3-OST and probed with fluorescent ATIII and washed. (a) Fluorescence image of ATIII bound to 3-OST-1 modified HS nanoparticles. (b) Transmitted bright field image of 3-OST-1 modified HS nanoparticles (c) Overlay of (a) and (b). (d) Fluorescence area selected by ImageJ from (a) shown as light spots on dark background. (e) Nanoparticle area selected by ImageJ from (b).

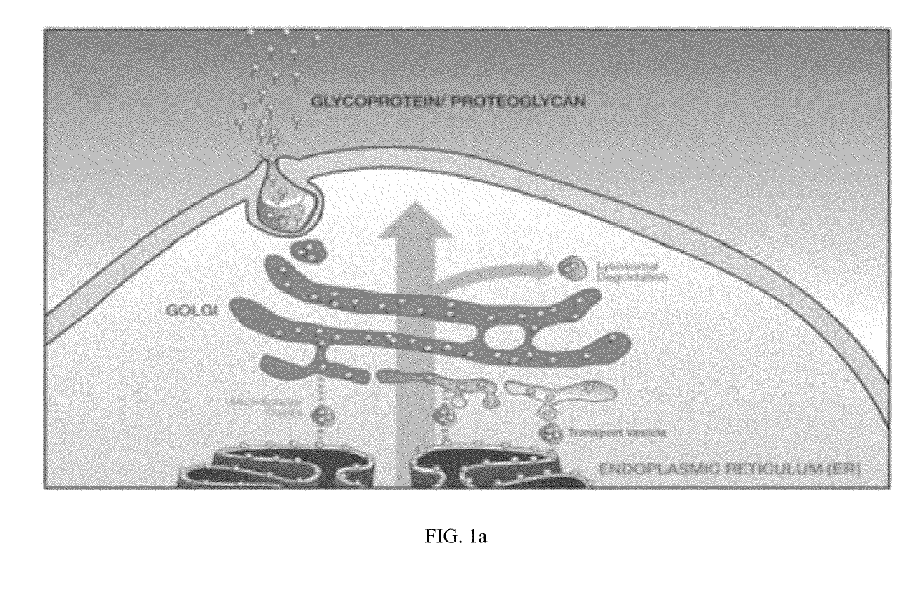

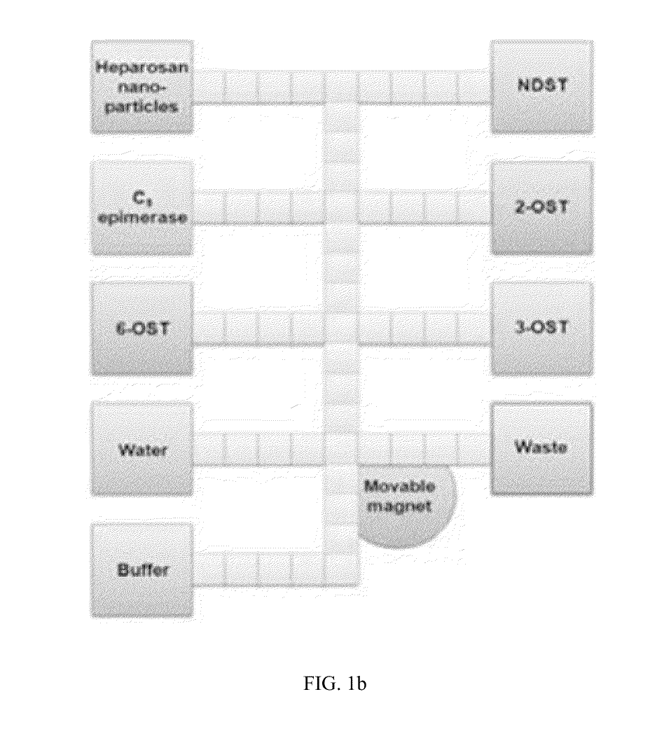

### DETAILED DESCRIPTION OF THE INVENTION

A description of preferred embodiments of the invention follows. The invention relates to an improved microfluidics device. Generally, microfluidics devices such as those described in U.S. Pat. No. 6,911,132 to Pamula et al., which is incorporated herein by reference in its entirety, can be used.

The present invention provides droplet-based liquid handling and manipulation methods by implementing electrowetting-based techniques. The droplets preferably contain magnetic nanoparticles with a substrate immobilized thereon. The droplets are preferably sub-microliter-sized, and can be moved freely by controlling voltages to electrodes. Generally, the actuation mechanism of the droplet is based upon surface tension gradients induced in the droplet by the voltage-induced electrowetting effect. The mechanisms of the invention allow the droplets to be transported while also acting as virtual chambers for mixing to be performed anywhere on a chip. The chip can include an array of electrodes that are reconfigurable in real-time to perform desired tasks. The invention enables several different types of handling and manipulation tasks to be performed on independently controllable droplet samples, reagents, diluents, and the like. These tasks include, for example, actuation or movement, monitoring, detection, irradiation, incubation, reaction, dilution, mixing, splitting, separation, dialysis, analysis, and the like.

Moreover, the methods of the invention can be used to form droplets from a continuous-flow liquid source, such as from a continuous input provided at a microfluidic chip. Accordingly, the invention provides a method for continuous sampling by discretizing or fragmenting a continuous flow into a desired number of uniformly sized, independently controllable droplet units, which can then be chemically and/or enzymatically modified in a controlled fashion.

The present invention manipulates a series of droplets on or in a microfluidic chip or other suitable structure by inducing and controlling electrowetting phenomena. The liquid is subsequently conveyed through or across the structure, preferably as a train of droplets.

The device and methods preferably comprise independently controlling the motion of each droplet. For purposes of chemical analysis, the sample droplets can be combined and mixed with droplets containing specific chemical reagents formed from reagent reservoirs on or adjacent to the chip or other structure. Multiple-step reactions or dilutions might be necessary in some cases with portions of the chip assigned to certain functions such as mixing, reacting or incubation of droplets. Once the sample is prepared, it can be transported by electrowetting to another portion of the chip dedicated to modification of the substrate. The substrate can be reacted with, for example, enzymes or other reagents. The flow of droplets can be controlled independently, allowing a great deal of flexibility.

Methods of the present invention comprise forming microdroplets, independently transporting, merging, and mixing droplets, maintaining droplets, particularly mixed droplets, under conditions suitable for modifying a substrate within the droplets, and other processing of the droplets. The preferred embodiment uses electrical control of surface tension (i.e., electrowetting) and substrate-immobilized magnetic nanoparticles to accomplish these manipulations.

In one embodiment, the liquid is contained within a space between two parallel plates. One plate contains the drive electrode broken into discrete squares on its surface while the other plate contains a single, continuous electrode that is grounded or set to a reference potential. Hydrophobic insulation covers the electrodes and an electric field is generated between electrodes on opposing plates. This electric field creates a surface-tension gradient that causes a droplet overlapping the energized discrete squares on the drive electrode resulting in its movement from square to square on that electrode. Through arrangement and control of the electrodes, a droplet can be transported by successively transferring it between adjacent squares on the drive electrode. The patterned squares on the drive electrode can be arranged in a two-dimensional array so as to allow transport of a droplet to one or more locations covered by that array. The space surrounding the droplets may be filled with a gas such as air or an immiscible fluid such as oil.

The structure used for ground or reference potential can be co-planar with the drive electrodes and the second plate, if used, can define the containment space. The co-planar grounding elements can be a conductive grid superimposed on the electrode array. Alternatively, the grounding elements can be electrodes of the array dynamically selected to serve as ground or reference electrodes while other electrodes of the array are selected to serve as drive electrodes.

Droplets can be combined together by transporting them simultaneously onto the same electrode. Droplets are subsequently mixed either passively or actively. Droplets are mixed passively by diffusion. Droplets are mixed actively by moving or “shaking” the combined droplet by taking advantage of the electrowetting phenomenon. In a preferred embodiment, droplets are mixed by rotating them around a two-by-two array of electrodes. The actuation of the droplet creates turbulent non-reversible flow, or creates dispersed multilaminates to enhance mixing via diffusion. Droplets can be split off from a larger droplet or continuous body of liquid in the following manner: at least two electrodes adjacent to the edge of the liquid body are energized along with an electrode directly beneath the liquid, and the liquid moves so as to spread across the extent of the energized electrodes. The intermediate electrode is then de-energized to create a hydrophobic region between two effectively hydrophilic regions. The liquid meniscus breaks above the hydrophobic regions, thus forming a new droplet. This process can be used to form the droplets from a continuously flowing stream.

According to one embodiment of the present invention, an apparatus for manipulating droplets comprises a substrate comprising a substrate surface, an array of electrodes disposed on the substrate surface, an array of reference elements, a dielectric layer disposed on the substrate surface, and an electrode selector. The reference elements are settable to a reference potential. The array of reference elements is disposed of in substantially co-planar relation to the electrode array, such that each reference element is adjacent to at least one of the electrodes. The dielectric layer is disposed on the substrate surface and is patterned to cover the electrodes. The electrode selector can be provided as a microprocessor or other suitable component for sequentially activating and de-activating one or more selected electrodes of the array to sequentially bias the selected electrodes to an actuation voltage. The sequencing performed by the electrode selector enables a droplet disposed on the substrate surface to move along a desired path that is defined by the selected electrodes.

According to one method of the present invention, a droplet is actuated by providing the droplet on a surface that comprises an array of electrodes and a substantially co-planar array of reference elements. The droplet is disposed on a first one of the electrodes, and at least partially overlaps a second one of the electrodes and an intervening one of the reference elements disposed between the first and second electrodes. The first and second electrodes are activated to spread at least a portion of the droplet across the second electrode. The first electrode can be de-activated to move the droplet from the first electrode to the second electrode.

The second electrode can be adjacent to the first electrode along a first direction. In addition, the electrode array can comprise one more additional electrodes adjacent to the first electrode along one or more additional directions. The droplet can at least partially overlap these additional electrodes as well as the second electrode. In accordance with this aspect of the method, the first direction that includes the first electrode and the second electrode is selected as a desired direction along which the droplet is to move. The second electrode is selected for activation based on the selection of the first direction.

A droplet can be split into two or more droplets and then, optionally, subjected to two or more distinct modifications in parallel. A starting droplet is provided on a surface comprising an array of electrodes and a substantially co-planar array of reference elements. The electrode array comprises at least three electrodes comprising a first outer electrode, a medial electrode adjacent to the first outer electrode, and a second outer electrode adjacent to the medial electrode. The starting droplet is initially disposed on at least one of these three electrodes, and at least partially overlaps at least one other of the three electrodes. Each of the three electrodes is activated to spread the starting droplet across the three electrodes. The medial electrode is de-activated to split the starting droplet into first and second split droplets. The first split droplet is disposed on the first outer electrode and the second split droplet is disposed on the second outer electrode.

In yet another method of the present invention, two or more droplets can be merged into one droplet. This is beneficial when mixing droplets with components of a reaction mixture, such as an enzyme composition and a substrate. First and second droplets are provided on a surface comprising an array of electrodes in a substantially co-planar array of reference elements. The electrode array comprises at least three electrodes comprising a first outer electrode, a medial electrode adjacent to the first outer electrode, and a second outer electrode adjacent to the medial electrode. The first droplet is disposed on the first outer electrode and at least partially overlaps the medial electrode. The second droplet is disposed on the second outer electrode and at least partially overlaps the medial electrode. One of the three electrodes is selected as a destination electrode. Two or more of the three electrodes are selected for sequential activation and de-activation, based on the selection of the destination electrode. The electrodes selected for sequencing are sequentially activated and de-activated to move one of the first and second droplets toward the other droplet, or both of the first and second droplets toward each other. The first and second droplets merge together to form a combined droplet on the destination electrode.

The apparatus for manipulating droplets can comprise a support comprising a support surface, an array of electrodes disposed on the surface, a dielectric layer disposed on the substrate surface and covering the electrodes, a hydrophobic layer, a top ground electrode coated with a hydrophobic layer and an electrode selector. The electrode selector dynamically creates a sequence of electrode pairs. Each electrode pair comprises a selected first one of the electrodes biased to a first voltage, and a selected second one of the electrodes disposed adjacent to the selected first electrode and biased to a second voltage that is less than the first voltage. Preferably, the second voltage is a zero ground voltage or some other reference voltage. A droplet disposed on the substrate surface moves along a desired path that runs between the electrode pairs created by the electrode selector.

Nanoparticles useful in the practice of the present invention are preferably magnetic (i.e., ferromagnetic) colloidal materials and particles. Magnetic particles include crosslinked SiO₂ colloids having magnetic materials encapsulated therein can also be used. Such nanoparticles are described in PCT WO/2005/052581 and U.S. patent application Publication No. 20050100930, which are incorporated herein by reference. The magnetic nanoparticles can be high moment magnetic nanoparticles which are small in size so as to be superparamagnetic, or synthetic antiferromagnetic nanoparticles which contain at least two layers of antiferromagnetically-coupled high moment ferromagnets. Both types of nanoparticles appear “nonmagnetic” in the absence of magnetic field, and do not normally agglomerate. In accordance with the present invention, magnetizable nanoparticles suitable for use comprise one or more materials selected from the group consisting of paramagnetic, superparamagnetic, ferromagnetic, and ferrimagnetic materials, as well as combinations thereof.

The nanoparticles can be high moment magnetic nanoparticles such as Co, Fe or CoFe nanocrystals which are superparamagnetic at room temperature. They can be fabricated by chemical routes such as salt reduction or compound decomposition in appropriate solutions. Examples of such magnetic nanoparticles have been published in the literature (S. Sun, and C. B. Murray, J. Appl. Phys., 85: 4325 (1999); C. B. Murray, et al., MRS Bulletin, 26: 985 (2001)). These particles can be synthesized with controlled size (e.g., 5 nm to 1 micron) and can be monodisperse.

The size of the magnetic nanoparticles suitable for use with the present invention is preferably such that the nanoparticles do not interfere with enzymatic processes. Consequently, the size of the magnetic nanoparticles is preferably from about 5 nm to about 1 micron, more preferably from about 20 nm to about 800 nm. Further, in addition to the more common spherical shape of magnetic nanoparticles, nanoparticles suitable for use with the present invention can be disks, rods, coils, or fibers.

Synthetic antiferromagnetic nanoparticles for use in this application may be considerably larger than ordinary ferromagnetic particles. This is because, to prevent clumping, the nanoparticle must have no net magnetic moment (or a very small magnetic moment) in zero applied field.

The substrates to be immobilized onto the nanoparticles include enzymatic substrates. The enzymes include proteases, peroxidases, lipases, carbohydrate cleavage enzymes, carbohydrases, esterases, carboxylases, peroxidases, nucleases, lyases, ligases, isomerases, transferases (e.g. NDST and OST), epimerases etc.

The enzymes are preferably those involved in the Golgi apparatus and include enzymes useful for the biosynthesis, conversion and modification of proteoglycans and other glycoproteins. The substrates are, therefore, preferably biomolecules, such as proteoglycans and heparan sulfate in particular. Other preferred substrates include native or modified proteoglycan core proteins that may be glycosylated in the Golgi apparatus, as well as glycoprotein core proteins. As used herein a “proteoglycan protein core” is a native or modified unglycosylated core protein (such as that synthesized by the ER in a cell) from which a proteoglycan is synthesized by glycosylation of the protein core. In accordance with the invention, the microfluidic device functions as an artificial Golgi apparatus capable of de novo biosynthesis of proteoglycans and other glycoproteins from proteoglycan core proteins and/or capable of specific modifications to all or any portion of one or more glucosaminoglycan chains of proteoglycans.

The enzymes are added to the substrate composition in an enzyme composition. The composition preferably contains any cofactors, buffers, stabilizers and the like desirable or necessary to conduct the reactions. Such cofactors, buffers and stabilizers are generally known in the art.

Other artificial organelles such as an Endoplasmic Reticulum (ER) could also be constructed using the same digital microfluidic device. In addition two artificial organelles could be combined and integrated on a single microfluidic device. For example, an artificial ER and an artificial Golgi could be integrated on a single device allowing the chemical and or enzymatic posttranslational modification and proper folding of protein to give functional glycoprotein or proteoglycan.

The planar electrode array of the digital microfluidics device are preferably coated with an oil, such as silicon oil, to reduce evaporation of the droplets and the non-specific adsorption of droplet contents to the device, thus preventing device fouling. Additionally, reducing the duration of the voltage application to minimize evaporation can also be beneficial.

The invention results in a novel a biomimetic system that provides an improved route to the synthesis of natural and unnatural glycosaminoglycans or proteoglycans in the case of a combined artificial Golgi-ER. Since glycosaminoglycans have important roles in many physiological and pathophysiological processes, they have great potential to be used for developing therapeutics, such as heparins free of animal contaminants. This device holds potential to develop custom glycosylation methods for the essential posttranslational modification of proteoglycan drugs.

The artificial Golgi of the present invention will provide an improved understanding of how glycosylation and other posttranslational modifications are controlled in the Golgi organelle. In addition, the artificial Golgi may be used as a test bed to design biosynthetic heparin, which could replace current unsafe methods of heparin production from animal tissue. Heparin is a life-saving multibillion-dollar drug and care must be taken to ensure a clean reliable supply.

### Experimental Section

Chip fabrication. Microfluidic chips were fabricated using standard clean room processes using a modified clean room process from Wheeler et al.¹⁰ Briefly, chips were designed using Macromedia Freehand (San Jose, Calif.) with standard dimensions of: electrode size (1 mm×1 mm), reservoir size (3 mm×3 mm), and electrode spacing (5 μm). Photomask transparencies constructed from these designs were made by M & J Prepress (Albany, N.Y.). Glass slides (Thermo Fisher Scientific, Waltham, Mass.) were cleaned with piranha solution (7:3 concentrated sulfuric acid/30% hydrogen peroxide, 10 min), followed by coating with 10 nm of chromium (International Advanced Materials, Spring Valley, N.Y.), and 100 nm of gold (International Advanced Materials Spring Valley, N.Y.) by electron beam deposition. The slides were then rinsed with acetone, methanol, and DI water and baked on a hotplate at 115° C. for 5 min. Substrates were spin coated with photoresist and exposed through a photomask using a Karl Suss mask aligner (Garching, Germany). The exposed substrates were developed in AZ300MIF (AZ Electronic Materials, Branchburg, N.J.) for 3 min and post-baked on a hotplate. The remaining unexposed gold and chromium was then etched away. The remaining photoresist was stripped in AZ300T (AZ Electronic Materials, Branchburg, N.J.) for 5 min in an ultrasonic bath. The chips were then coated with 2 μm parylene C followed by spin-coating with a 1% Teflon AF solution in FC-40 (DuPont, Wilmington, Del.) at 2000 rpm for 1 min. The extra solvent was then evaporated by placing the chip on a hotplate at 160° C. for 10 min. Indium-tin-oxide coated glass slides (Delta Technologies, Stillwater, Minn.) were also coated with a 1% Teflon AF solution and used as coverslips.

Chip operation. Electrical equipment used for chip operation included an Agilent 33220A 20 MHz Function/Arbitrary Waveform Generator (Santa Clara, Calif.) and a Trek Model PZD700 Dual Channel Amplifier (Medina, N.Y.). Four basic droplet operations for digital microfluidic devices were used in this study: 1) droplet actuation; 2) dispensing; 3) splitting; and 4) mixing. Droplet actuation was achieved by applying an AC voltage 50-100 V_{RMS} to a destination electrode adjacent to the droplet. Sample droplets were dispensed from reservoir droplets by applying voltage to electrodes adjacent to the reservoir to draw a portion of the droplet away from the reservoir while holding the remaining bulk of the droplet within the reservoir using a separate application of voltage. After a distance of about two electrodes, surface tension caused splitting of the sample droplet from the reservoir droplet. Droplets were split by application of voltage to the electrodes adjacent to the ends of the droplet and maintaining the voltage until the splitting occurred. Droplets were mixed by actuating two droplets to the same electrode then homogenized by pulsing voltage to the electrodes adjacent to the ends of the droplet to cause stretching.

Immobilization of heparan sulfate onto magnetic nanoparticles. Sodium HS from porcine intestinal mucosa was obtained from Celsius Laboratories (Cincinnati, Ohio). The HS was first completely N-acetylated at 25° C. by dissolving 200 mg of HS in 10 mL 0.05 M sodium carbonate (Sigma-Aldrich, St. Louis, Mo.) containing 10% methanol (Sigma-Aldrich, St. Louis, Mo.) and adding 80 μL acetic anhydride (Sigma-Aldrich) dropwise over a period of 2 h. The solution containing N-acetylated HS was then diluted with two volumes of distilled water, dialyzed (2,000 molecular weight cut-off (MWCO)) for 48 h and lyophilized. This fully N-acetylated heparin (100 mg) was dissolved in formamide (8.3 μM) (Sigma-Aldrich) at 50° C., 2,6-diaminopyridine (100 mg) (Sigma-Aldrich) was added, and the reaction mixture was maintained at 50° C. for 3 h. Sodium cyanoborohydride (19 mg) (Sigma-Aldrich) was added and the mixture was maintained at 50° C. for 24 h, diluted with 20 mL of water, dialyzed (2,000 MWCO) exhaustively for 48 h, and lyophilized. The lyophilized diaminopyridylinylated HS was further purified by SAX chromatography followed by methanol precipitation.¹¹ The resulting 2,6-diaminopyridinyl heparin was dissolved in phosphate buffered saline (PBS, pH 7.4) and a 20-fold molar excess of Sulfo-NHS-PEG-Biotin (5000 MW) (Nanocs, New York, N.Y.) was added. The reaction mixture was incubated at 25° C. for 1 h and then dialyzed (3,000 MWCO) to remove excess biotin. The retentate containing biotinylated HS was then lyophilized.

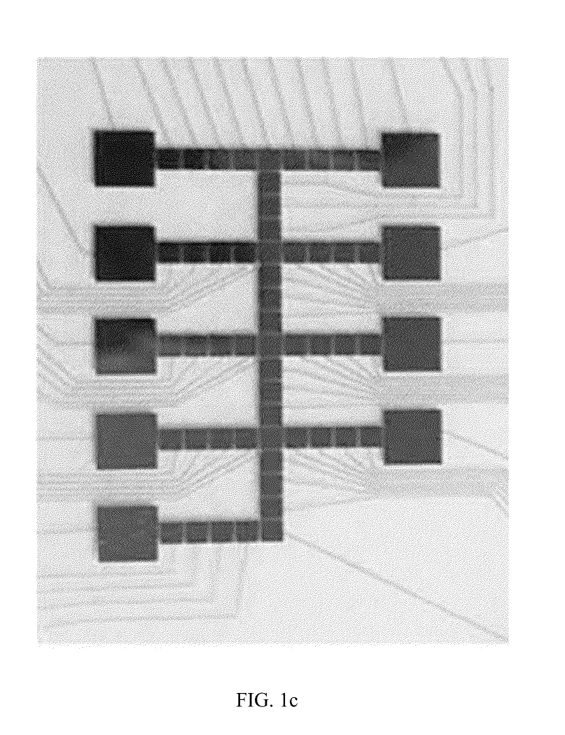

Using a magnetic rack (New England Biolabs, Ipswitch, Mass.), streptavidin magnetic nanoparticles (600 nm diameter) from MagnaMedics Diagnostics (Maastricht, The Netherlands) were washed with 0.05% Triton X-100 (Sigma-Aldrich) in PBS. Biotiylated HS was added (3-fold molar excess) to the solution of streptavidin magnetic nanoparticles and the mixture was incubated on a roller mixer at 25° C. for 45 min. The nanoparticles were then washed extensively with 0.05% Triton X-100 in PBS to remove the excess biotinylated HS. The nanoparticles were further washed with PBS buffer to remove the Triton X-100 surfactant. The overall immobilization scheme is shown in FIG. 2.

Quantification of heparan sulfate on magnetic nanoparticles by enzymatic digestion. HS-nanoparticles (1 mg) were incubated with 25 milliunits each of a mixture of heparin lyase 1, 2, and 3 for 24 h at 37° C. to quantify the amount of HS that had been immobilized. After 24 h, the nanoparticles were isolated using the magnetic rack and the supernatant was recovered by decanting. The supernatant, containing heparin lyases and HS disaccharides, was passed through a 3,000 MWCO spin column (PALL, East Hills, N.Y.) to recover the free HS disaccharides from the enzymes. A microscale carbazole assay was used to determine the amount of free disaccharides present from which the amount of enzyme accessible immobilized HS chains could be calculated.

Quantification of modification of heparan sulfate on nanoparticles by 3-OST. Recombinant 3-OST-1 enzyme was prepared as previously described¹² and adenosine 3′-phosphate 5′-phosphosulfate (PAPS) was obtained from Sigma-Aldrich (St. Louis, Mo.). [³⁵S]PAPS was prepared from ATP and sodium [³⁵S] sulfate in the presence of yeast extract (from Sigma-Aldrich).¹³ Immobilized HS (about 20 μg) was incubated with purified 3-OST-1 (300 μg) and PAPS (100 mM) in 300 mL of reaction buffer containing 50 mM MES (pH 7.0), 1 mM MgCl₂, and 2 mM MnCl₂. The reaction was shaken at 30° C. at 200 RPM for 2 h. The nanoparticles were then washed with 3×1 mL of 250 mM NaCl followed by 3×1 mL of 1 M NaCl. An identical reaction was carried out by replacing unlabeled PAPS with ³⁵S-labeled PAPS to estimate the degree of 3-O-sulfonation. The degree of 3-O-sulfonation was estimated by measuring ³⁵S-radioactivity on the nanoparticles. A time course reaction was carried out by incubating immobilized HS (50 μg) with purified 3-OST-1 (100 μg) and [³⁵S]PAPS at 30° C. The reaction was stopped periodically and an aliquot was removed, and the beads were washed with buffer containing 3 M urea followed by buffer with 1 M NaCl. The washed beads were then mixed with scintillation fluid to determine the level of incorporation of ³⁵S-label.

Synthesis of Fluorescently labeled ATIII. Amine reactive 4,4-Difluoro-5-phenyl-4-bora-3,4a-diaza-s-indacene-3-propionic acid, succinimidyl ester (BODIPY® R6G, SE) was obtained from Invitrogen Corporation (Carlsbad, Calif.). ATIII was obtained from Aniara (Mason, Ohio). BODIPY® R6G dye was covalently attached to ATIII. ATIII (10 mg) was dissolved in 1 mL of 0.1 M sodium bicarbonate buffer and BODIPY® R6G (5 mg) was dissolved in 0.5 mL anhydrous DMF (Sigma-Aldrich, St. Louis, Mo.). BODIPY® R6G (100 μL) was added with stirring to the ATIII solution. The reaction mixture was incubated for 1 h at room temperature with continuous stirring. After the completion of the reaction, a 3,000 MWCO spin column was used to remove excess BODIPY® R6G. The purified BODIPY® R6G labeled ATIII was then stored at −20° C.

Off-chip enzymatic modification. PAPS and 3-OST-1 were mixed with nanoparticles in PBS then washed with distilled water as described above. The 3-OST-1 modified nanoparticles (100 μg) were incubated at 25° C. with BODIPY® R6G labeled ATIII (1 μg) in 10 mL of 2.5-fold concentrated PBS containing 0.025% Tween X-100 for 30 min. After incubation, the nanoparticles were washed with 0.05% Tween X-100 in 5-fold concentrated PBS then distilled water. The nanoparticles were imaged using a Zeiss LSM 510 META confocal microscope with a 100×/1.45 oil objective (Zeiss, Jena, Germany). A 514 nm argon laser was used to excite BODIPY® R6G and a 530 nm-600 nm emission filter was used. A range check pseudocolored map was used to optimize the dynamic range for the images.

On-chip enzymatic modification. Two droplets, one containing 400 nL of 3-OST-1 (0.1 mg/mL) and PAPS (25 μM) in PBS and the second containing nanoparticle-immobilized HS (5 mg/mL) in PBS, were loaded onto a digital microfluidics chip into a layer of 1.0 cSt silicone oil (Gelest, Morrisville, Pa.) containing 0.1% Triton® X-15 (Sigma-Aldrich, St. Louis, Mo.). The use of silicon oil and the short duration of electrode activation reduced non-specific adsorption of protein to the surface and droplet heating. The chip was used to drive the droplets to the mixing area and perform the mixing operation by first joining the two droplets then stretching the droplet to homogenize the reaction mixture. Following the mixing operation, the reaction droplet was incubated at room temperature for 1 h with an additional homogenizing step at 30 min. Based on the results of the 3-OST-1 time course experiment (see Supporting Information FIG. S1), an on-chip incubation time of 1 h was selected to allow for at least one 3-O-sulfonation site per HS chain, as is present in the natural polysaccharide. After the incubation was complete, the droplet was recovered using a micropipette added to an 800 nL droplet of water following addition of a 400 nL droplet containing fluorescent ATIII (0.175 mg/mL) and 0.0375% Tween 100 in 3.75-fold concentrated PBS. The 3-OST-1 modified HS-nanoparticles were incubated with fluorescent ATIII at room temperature for 30 min. After incubation, the nanoparticles were washed with 0.05% Tween X-100 in 5-fold concentrated PBS then distilled water. The nanoparticles were imaged using a Zeiss LSM 510 META confocal microscope with a 100×/1.45 oil objective (Zeiss, Jena, Germany). A 514 nm argon laser was used to excite BODIPY® R6G and a 530 nm-600 nm emission filter was used. A range check pseudocolored map was used to optimize the dynamic range for the images. Control experiments were also conducted using a 400 nL droplet of PBS in place of the 3-OST-1 enzyme in PBS.

Analysis of confocal images. Transmitted bright field images acquired from confocal microscopy were imported in to Adobe Photoshop (Adobe Systems Incorporated, San Jose, Calif.) where the background was selected and filled with white.

The images were then imported into ImageJ (National Institutes of Health, Bethesda, Md.) and converted to 16-bit grayscale. The ImageJ analyze particle feature was used to determine the total area of the nanoparticles. Next, the confocal fluorescence images were converted to 16-bit grayscale images and imported into ImageJ. The images were processed using the despeckle feature and all pixels of low intensity (2 out of 255 or lower) were ignored to remove noise. The analyze particle feature in ImageJ was used to determine the total area of the fluorescent ATIII on the nanoparticles. The total area was then used to calculate the percent coverage of fluorescent ATIII on the nanoparticles.

### Results

Immobilization and functional properties of heparan sulfate bound to magnetic nanoparticles. Biotinylated HS was successfully prepared and immobilized onto streptavidin coated magnetic nanoparticles (FIG. 2). The amount of enzyme-accessible HS, determined using heparinase, was 2.7 μg/mg of nanoparticles. This amount reflects the minimum amount biotinylated HS attached to the nanoparticles, which represents only the enzyme-accessible HS.

The activity of 3-OST-1 on HS-linked nanoparticles was determined using [³⁵S]PAPS and scintillation counting. 3-OST-1 (120 μg) catalyzed the transfer of 4 pmol of 3-O-sulfo groups to 8.1 μg of HS linked to nanoparticles (FIG. 2), which elevated the ³⁵S-radioactivity of the enzymatically modified particles to ˜70-fold above the control (absence of 3-OST-1 enzyme). This corresponded to the introduction of a ³⁵S-sulfo group onto ˜5% of the immobilized HS chains. This value is expected based on the specificity of 3-OST-1 for the limited number of modifiable domains present within HS.¹⁴

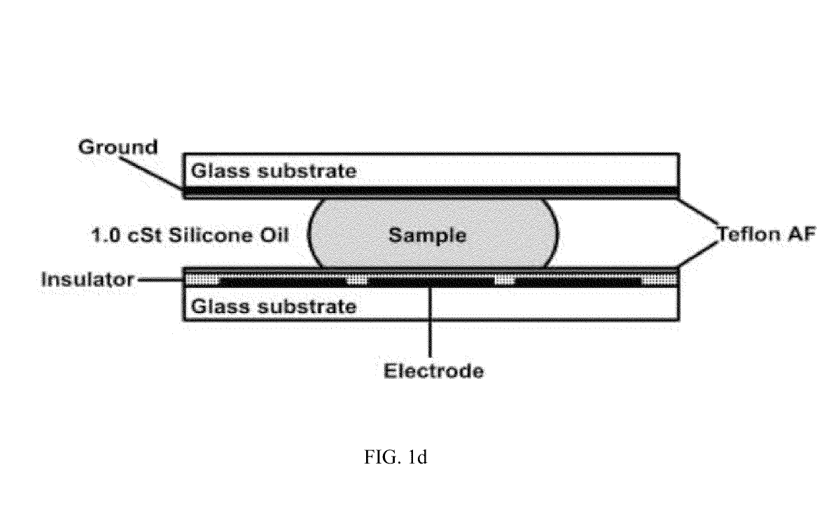

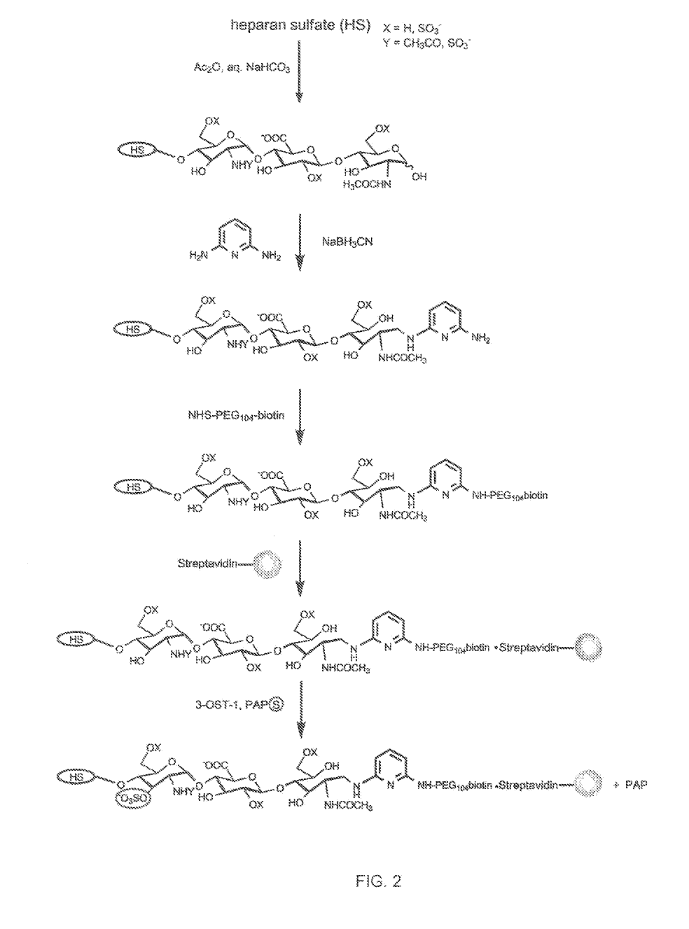

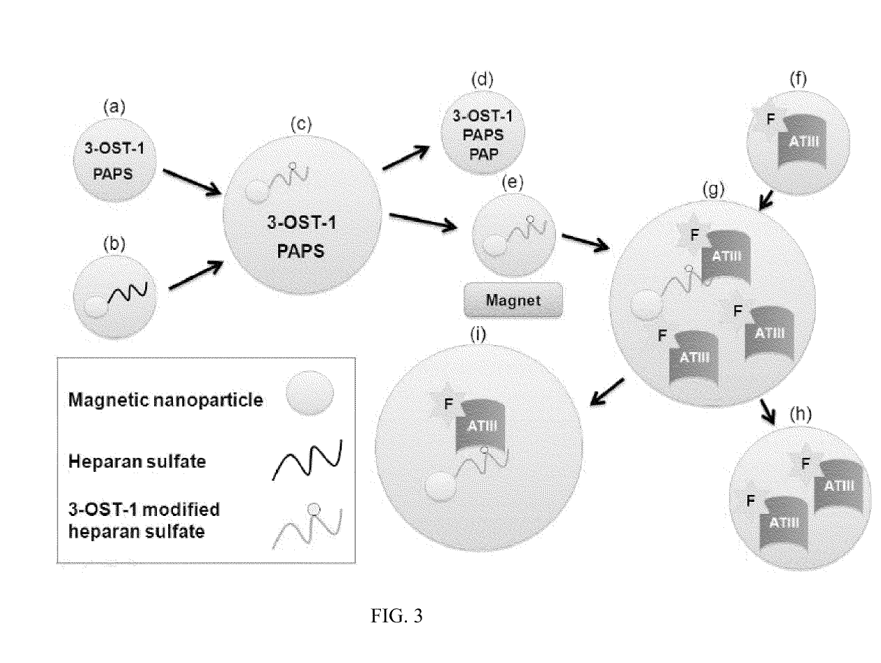

After confirming that HS immobilized onto the nanoparticles was capable of being modified by 3-OST-1, we performed modification of immobilized HS on a new set of nanoparticles without using radiolabeling. We then removed the 3-OST-1 from the reaction mixture by precipitating the magnetic nanoparticles and incubated the modified nanoparticles with fluorescent ATIII to investigate the binding properties of the 3-OST-1 modified immobilized HS (FIG. 3).

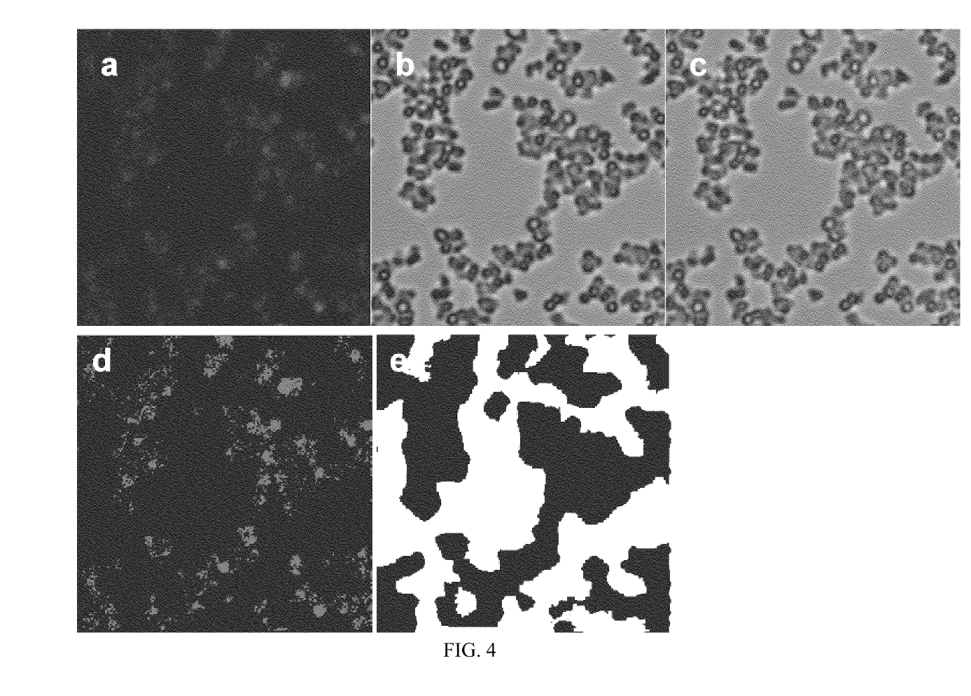

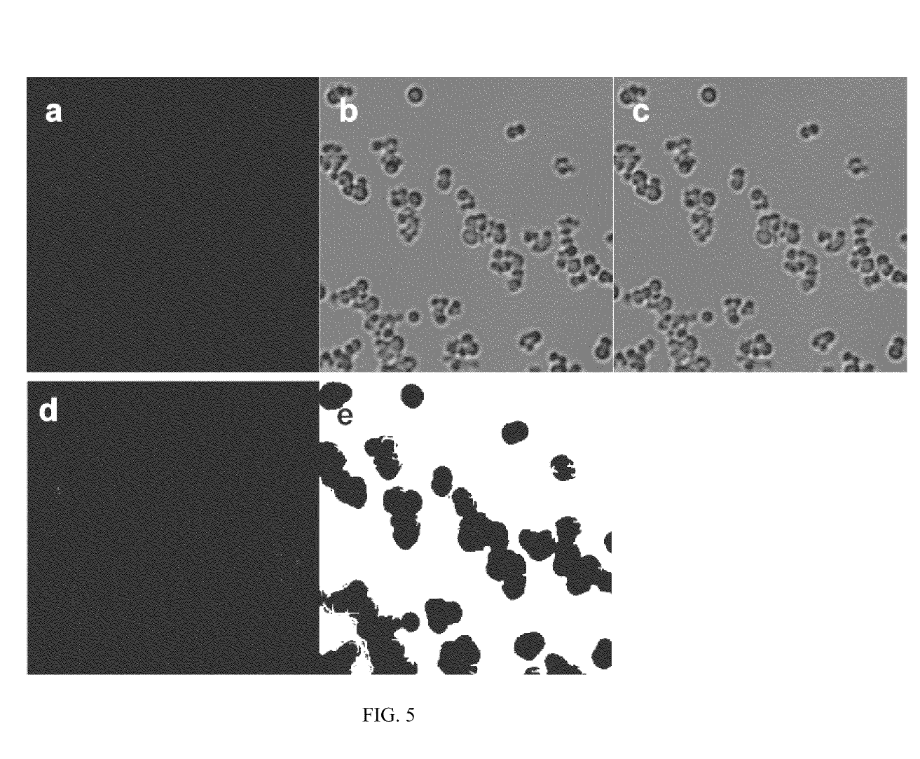

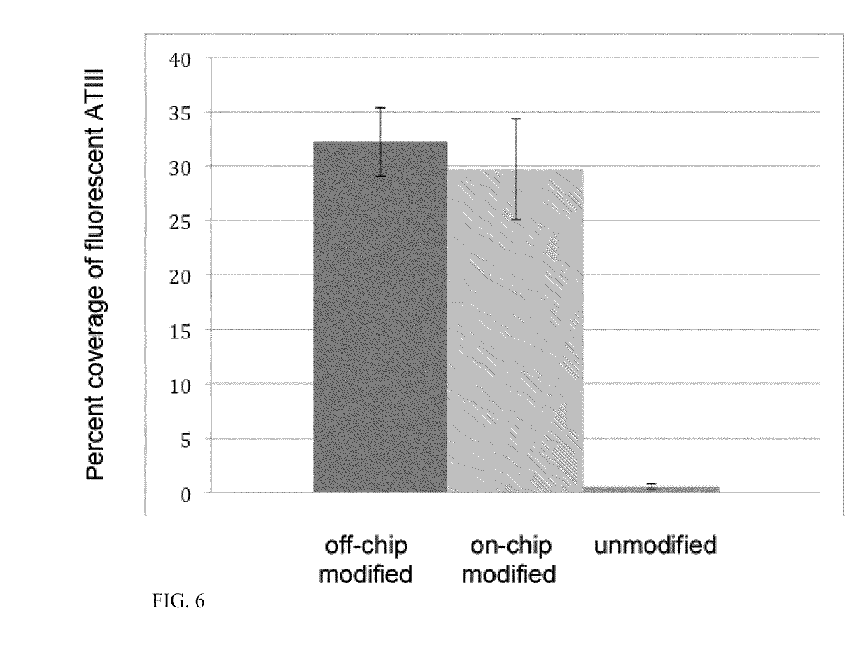

Using confocal microscopy, a pronounced increase in the binding of fluorescent ATIII was observed following 3-OST-1 modification of immobilized HS on nanoparticles (FIG. 4 and Supporting Information FIG. S2) as compared to unmodified immobilized HS on nanoparticles (FIG. 5 and Supporting Information FIG. S2). The binding is specific to the modified HS nanoparticles as clearly seen in FIG. 4c, the overlay of the fluorescence image (FIG. 4a) and the transmitted bright field image (FIG. 4b). Analysis using ImageJ software, demonstrated that 32% of the surface area of the 3-OST modified HS nanoparticles bound fluorescent ATIII (FIG. 4, d & e and FIG. 6), whereas only 0.5% of the surface area of the control unmodified HS nanoparticles bound fluorescent ATIII (FIG. 5, d & e and FIG. 6).

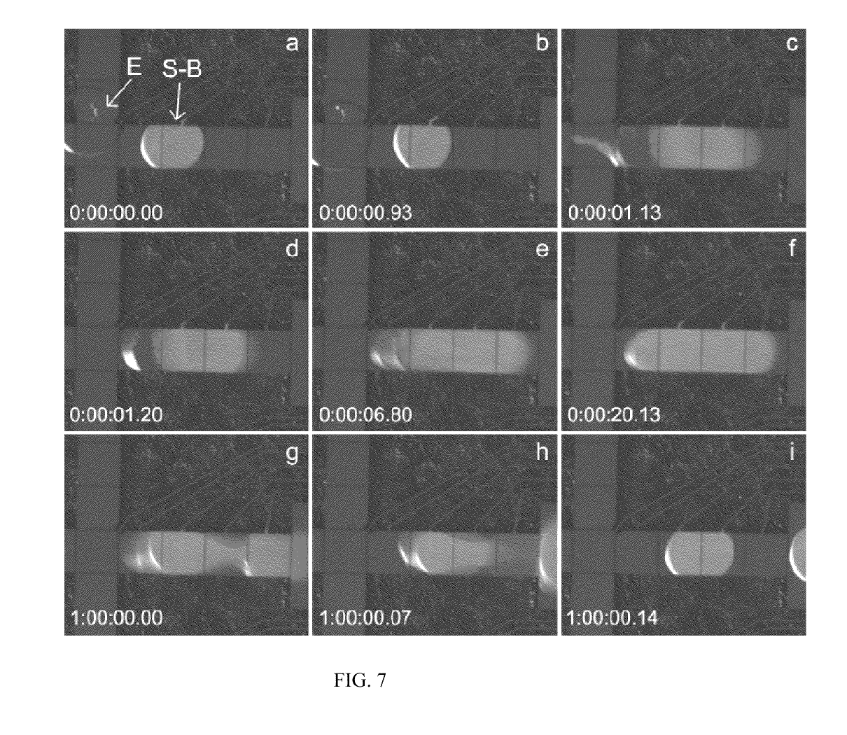

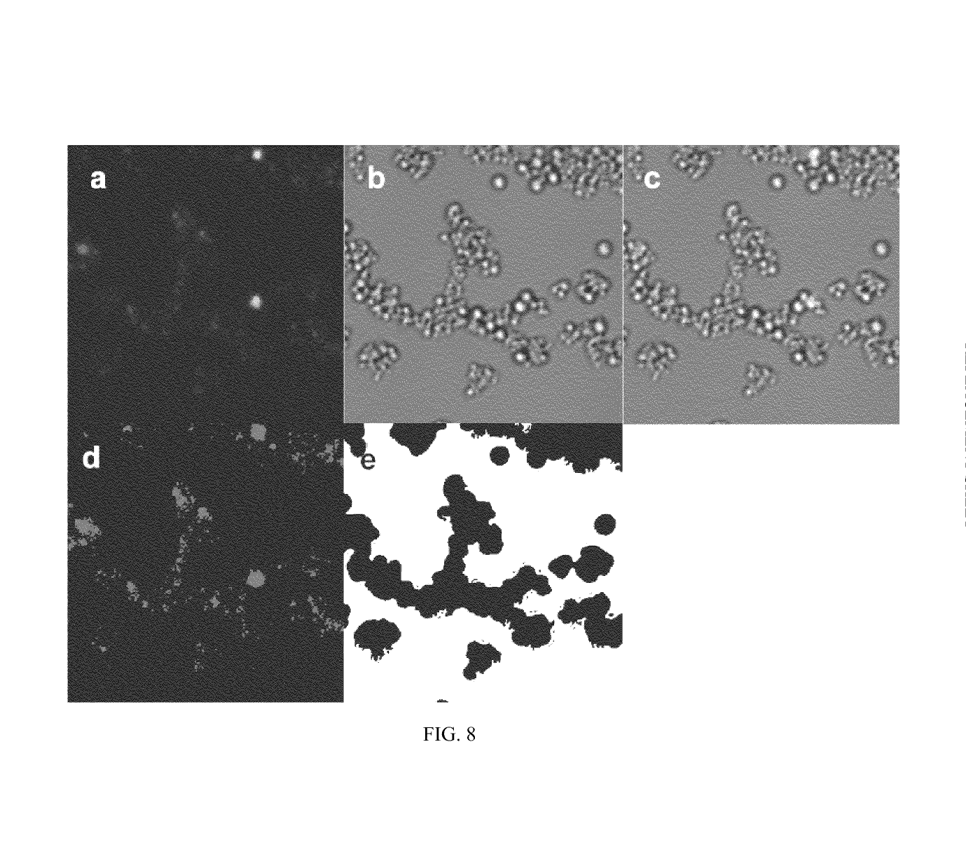

On-chip modification of heparan sulfate on magnetic nanoparticles and subsequent protein binding assay of modified heparan sulfate. The ability to detect ATIII binding resulting from 3-OST-1 catalyzed modification of HS nanoparticles led us to perform on-chip reactions. Using the digital microfluidic chip, two droplets—one containing 3-OST-1 (40 ng, 1 pmol) and PAPS cofactor (25 μM) and the second containing HS immobilized onto nanoparticles (5.4 ng, 0.4 pmol)—were loaded onto the chip. The chip was then used to drive the droplets to a mixing area by application of an AC voltage of 50-100 V_{RMS} to the electrode adjacent to the droplet. The droplets were able to move at a rate of up to 3 electrodes per min, corresponding to about 3 mm per min. Once at the mixing area, the droplets were merged by driving them to the same destination electrode (FIG. 7, a-c), followed by homogenizing the reaction droplet by applying voltage (FIG. 7, d-f). The reaction droplet was allowed to incubate on the chip for a period of 1 h with an additional homogenizing step at 30 min to keep the nanoparticles in suspension. Following the reaction, we performed a splitting of the reaction droplet to show the potential for additional reactions to be performed in a combinatorial manner (FIG. 7, g-iand Supporting Information FIG. S2), but then proceeded to rejoin the droplet. The droplets were then recovered from the chip, incubated with fluorescent ATIII, washed to remove excess fluorescent ATIII, and observed using confocal fluorescent microscopy (FIG. 8). The results were similar to the experiments performed off-chip (FIG. 4) showing a significant increase in the ATIII binding affinity of the 3-OST-1 modified HS nanoparticles when compared to the unmodified control. Analysis using ImageJ software, demonstrated that 30% of the surface area of the on-chip 3-OST modified HS nanoparticles bound fluorescent ATIII (FIG. 6 and FIG. 8d & e), whereas only 0.5% of the surface area of the control unmodified HS nanoparticles bound fluorescent ATIII (FIG. 5d & e and FIG. 6). These results confirm that 3-OST-1 can be used to successfully modify nanoparticle immobilized HS in a digital microfluidic system, resulting in HS chains with high ATIII affinity.

### Discussion

The loading of HS onto the magnetic nanoparticles was about 20 percent of the manufacturers projected loading capacity, and can be explained by the large steric size of HS. According to manufacturer's specifications, the loading capacity of the streptavidin nanoparticles for the small molecule of biotin-4-fluorescein (MW 645 Da) is 1000 pmol per mg of nanoparticles. We measure a loading of about 200 pmol of HS (average MW 12500 Da) per mg of nanoparticles based on an enzymatic digestion assay.

When the activity of 3-OST-1 on HS-linked nanoparticles was determined by [³⁵S] PAPS and scintillation counting, it was observed that the level of 3-O-sulfation was about 70-fold higher on the 3-OST-1 modified nanoparticles compared to the control. Using these values we calculated that 3-OST-1 catalyzed the introduction of a ³⁵S-sulfo group to about 5% of the immobilized HS chains. This value is low because not all HS chains contain an ATIII precursor structure¹⁴ and 3-OST-1 may not have been able to access all of the immobilized HS, e.g., those buried from the surface of the exposed HS chains on the nanoparticles. However, even though the sulfation level was low, there was still an easily measurable increase in HS's binding affinity for ATIII, as detected by confocal microscopy (FIGS. 4, 5 and 8). Results from the on-chip and off-chip experiments were comparable; 32% of the surface area of the off-chip 3-OST modified HS nanoparticles bound fluorescent ATIII and 30% of the surface area of the on-chip 3-OST modified HS nanoparticles bound fluorescent ATIII (FIG. 6). In the images obtained, virtually all the beads from both on-chip and off-chip experiments showed bound fluorescent ATIII (FIGS. 4 and 8c). As expected, not all of the surface area was covered with fluorescent ATIII, as only 5% of the chains could be modified using 3-OST-1, based on steric factors and the limited number of modifiable domains within HS. The control experiments (FIG. 5 and Supporting Information FIG. S2) showed virtually no measureable fluorescence associated with non-specific binding. Image analysis demonstrates experimental (specific binding) is ˜50-fold greater than control (non-specific binding) (FIG. 6).

In summary, this work represents a first step toward the construction of an artificial Golgi organelle through the use of digital microfluidics, recombinant enzyme technology, and magnetic nanoparticles. HS was successfully immobilized onto magnetic nanoparticles and enzymatically modified using 3-OST-1 in a droplet based digital microfluidic device. After enzymatic treatment, the immobilized HS showed high affinity for fluorescent ATIII, confirming its successful modification by 3-OST-1. This represents the first enzymatic modification of an immobilized substrate in a digital microfluidic device and a critical first step in the creation of an artificial Golgi. Work is currently underway to further develop the artificial Golgi into a nano-scale lab-on-a-chip for the synthesis and screening of specific glycans. Such an artificial Golgi will provide a platform for the high throughput synthesis of small amounts of GAGs for biological and pharmacological evaluation. In addition, this tool may be used as a test bed to design biosynthetic heparin, which could replace current unsafe methods of heparin production from animal tissue.¹⁵

While this invention has been particularly shown and described with references to preferred embodiments thereof, it will be understood by those skilled in the art that various changes in form and details may be made therein without departing from the scope of the invention encompassed by the appended claims.

### REFERENCES

(1) Twyman, R. M. Principles of Proteomics. New York, NY: Garland Science/BIOS Scientific, 2004.

(2) Munoz, E., Xu, D., Kemp, M., Zhang, F. & Liu, J. Biochemistry 45, 5122-5128 (2006).

(3) Esko, J. D., Selleck, S. B., Ann. Rev. Biochem. 2002, 71, 435-471.

(4) Sasisekharan, R. & Venkataraman, G, Curr. Opin. Chem. Biol. 2000, 4, 626-631.

(5) Capila, I. & Linhardt, R.J., Angew. Chem. Int. Ed. 2002, 41, 390-412.

(6) Srinivasan, V., Pamula, V. & Fair, R., Lab Chip 2004, 4, 310-315.

(7) Wheeler, A. R., Science 2008, 322, 539-540.

(8) (a) Srinivasan, V., Pamula, V. & Fair, R., Anal. Chim. Acta 2004, 507, 145-150. (b) Miller E. M. & Wheeler A. R., Anal. Chem. 2008, 80, 1614-1619. (c) Moon, H., Wheeler, A. R., Garrell, R. L., Loo, J. A. & Kim, C. J., Lab Chip 2006, 6, 1213-1219. (d) Chang, Y. H., Lee, G. B., Huang, F. C., Chen, Y. Y. & Lin, J. L., Biomed. Microdevices 2006, 8, 215-225. (e) Barbulovic-Nad, I., Yang, H., Park, P. & Wheeler, A. R. Lab Chip 2008, 8, 519-526.

(9) Wheeler, A. R. Science 2008, 322, 539-540.

(10) Watson, M. W., Abdelgawad, M., Ye, G., Yonson, N., Trottier, J., Wheeler, A. R. Anal. Chem. 2006, 78, 7877-7885.

(11) Nadkarni, V. D. & Linhardt, R. J. Anal. Biochem. 1994, 222, 59-67.

(12) Edavettal, S. C., Lee, K. A., Negishi, M., Linhardt, R. J., Liu, J., Pedersen, L. C. J. Biol. Chem. 2004, 279, 25789-25797.

(13) Bame, K. J. & Esko, J. D. J. Biol. Chem. 1989, 264, 8059-8065.

(14) Linhardt, R. J. Wang, H. M. Loganathan, D. Bae, J. H. J. Biol. Chem. 1992, 267, 2380-2387.

(15) Guerrini, M., et al., Nat. Biotechnol. 2008, 26, 669-675.

1. A method for enzymatically producing a biological compound from a substrate comprising,
(1) providing a digital microfluidics device comprising:
(a) a support comprising a support surface; (b) an array of drive electrodes disposed on the surface; and (c) an electrode selector for sequentially activating and de-activating one or more selected drive electrodes of the array to sequentially bias the selected drive electrodes to an actuation voltage, whereby a droplet disposed on the substrate surface moves along a desired path defined by the selected drive electrodes;
(2) providing a first droplet comprising a substrate, immobilized on a magnetic nanoparticle;
(3) providing an enzyme composition, in a second droplet;
(4) contacting the first droplet and enzyme composition under conditions suitable for reacting the enzyme composition with the substrate;
(5) optionally separating the magnetic nanoparticles using a magnet; and
(6) activating and/or deactivating said drive electrodes to transport the droplet, or a portion thereof

2. The method of claim 1, further comprising the steps of providing a second enzyme composition in a third droplet, and contacting the reaction product of step (4) with the second enzyme composition under conditions suitable for reacting the second enzyme composition with the reaction product.

3. The method of claim 2, wherein the microfluidics device is an artificial Golgi apparatus.

4. The method of claim 2, wherein the microfluidics device is an artificial ER.

5. The method of claim 2, wherein the microfluidics device is a combined artificial ER and artificial Golgi apparatus.

6. The method of claim 1, wherein the substrate is heparan sulfate.

7. The method of claim 1, wherein the droplet is deposited onto the microfluidics device on a layer of silicon oil.

8. The method of claim 1, wherein the electrode activation is selected to avoid droplet heating and/or evaporation.

9. A magnetic composition comprising a magnetic nanoparticle having immobilized thereon an enzymatic substrate.

10. The composition of claim 9, wherein the magnetic nanoparticle has a particle size of between 5 nm and 1 micron.

11. The composition of claim 10, wherein the enzymatic substrate is a proteoglycan, or glycoprotein or heparin sulfate.

12. The composition of claim 10, wherein the enzymatic substrate is heparan sulfate.

13. (canceled)

14. A method for modifying a proteoglycan comprising:
(1) providing a digital microfluidics device comprising:
(a) a support comprising a support surface; (b) an array of drive electrodes disposed on the surface; and (c) an electrode selector for sequentially activating and de-activating one or more selected drive electrodes of the array to sequentially bias the selected drive electrodes to an actuation voltage, whereby a droplet disposed on the substrate surface moves along a desired path defined by the selected drive electrodes;
(2) providing a first droplet on the microfluidic device comprising a proteoglycan substrate immobilized on a magnetic nanoparticle;
(3) providing a second droplet on the microfluidic device comprising an enzyme composition capable of modifying at least one glycosaminoglycan present on the proteoglycan substrate;
(4) contacting the first droplet with the second droplet under conditions suitable for reacting the enzyme composition of the second droplet with the proteoglycan substrate of the first droplet, wherein the reaction results in the modification of all or a portion of at least one glycosaminoglycan present on the proteoglycan substrate, wherein the contacting is the result of activating and/or deactivating said drive electrodes to cause the first droplet and the second to come in contact; and
(5) optionally separating the magnetic nanoparticles using a magnet.

15. A method for synthesizing a proteoglycan from a proteoglycan protein core comprising:
(1) providing a digital microfluidics device comprising:
(a) a support comprising a support surface; (b) an array of drive electrodes disposed on the surface; and (c) an electrode selector for sequentially activating and de-activating one or more selected drive electrodes of the array to sequentially bias the selected drive electrodes to an actuation voltage, whereby a droplet disposed on the substrate surface moves along a desired path defined by the selected drive electrodes;
(2) providing a first droplet on the microfluidic device comprising a proteoglycan core protein substrate immobilized on a magnetic nanoparticle;
(3) providing a second droplet on the microfluidic device comprising an enzyme composition capable of facilitating glycosylation of the proteoglycan protein core;
(4) contacting the first droplet with the second droplet under conditions suitable for reacting the enzyme composition of the second droplet with the proteoglycan substrate of the first droplet, wherein the reaction results in the glycosylation of proteoglycan core protein substrate, wherein the contacting is the result of activating and/or deactivating said drive electrodes to cause the first droplet and the second to contact; and
(5) optionally separating the magnetic nanoparticles using a magnet.

16. The method according to claim 15 wherein at least a portion of the product of step (4) is directed to and contacted with a third droplet comprising an enzyme composition capable of modifying the product under conditions suitable for reacting the enzyme composition with the product.

17. The method according to claim 15 wherein a portion of the product of step (4) is directed to and contacted with a third droplet comprising an enzyme composition capable of modifying the product under conditions suitable for reacting the enzyme composition with the product and a portion of the product of step (4) is directed to and contacted with a fourth droplet comprising an enzyme composition capable of modifying the product under conditions suitable for reacting the enzyme composition with the product.

18. The method according to claim 14 wherein at least a portion of the product of step (4) is directed to and contacted with a third droplet comprising an enzyme composition capable of modifying the product under conditions suitable for reacting the enzyme composition with the product.

19. The method according to claim 14 wherein a portion of the product of step (4) is directed to and contacted with a third droplet comprising an enzyme composition capable of modifying the product under conditions suitable for reacting the enzyme composition with the product and a portion of the product of step (4) is directed to and contacted with a fourth droplet comprising an enzyme composition capable of modifying the product under conditions suitable for reacting the enzyme composition with the product.

20. The method according to claim 1 wherein at least a portion of the product of step (4) is directed to and contacted with a third droplet comprising an enzyme composition capable of modifying the product under conditions suitable for reacting the enzyme composition with the product.

21. The method according to claim 1 wherein a portion of the product of step (4) is directed to and contacted with a third droplet comprising an enzyme composition capable of modifying the product under conditions suitable for reacting the enzyme composition with the product and a portion of the product of step (4) is directed to and contacted with a fourth droplet comprising an enzyme composition capable of modifying the product under conditions suitable for reacting the enzyme composition with the product.
